(19) United States
(12) Patent Application Publication
(10) Pub. No.: US 2009/0264756 A1
(43) Pub. Date: Oct. 22, 2009

(54) TISSUE THICKNESS AND STRUCTURE MEASUREMENT DEVICE

(75) Inventors: Luiz B. DA SILVA, Danville, CA (US); Drew A. STARK, Livermore, CA (US)

Correspondence Address: LEVINE BAGADE HAN LLP, 2400 GENG ROAD, SUITE 120, PALO ALTO, CA, 94303, US

(73) Assignee: IntelaMetrix Inc., Livermore, CA (US)

(21) Appl. No.: 12/434,593

(22) Filed: May 1, 2009

Related U.S. Application Data
(63) Continuation of application No. PCT/US2007/083480, filed on Nov. 2, 2007.
(60) Provisional application No. 60/856,112, filed on Nov. 2, 2006.

(51) Int. Cl. A61B 8/00

(52) U.S. Cl. 600/442

(57) ABSTRACT

A device is described that can be easily used to accurately measure and monitor tissue thickness and structure using ultrasound. The device comprises a remote control and data processing unit, a handheld ultrasound transducer with an integrated position sensor.

### CROSS-REFERENCE TO RELATED APPLICATIONS

This application is a continuation of International Application Number PCT/US2007/083480 filed Nov. 2, 2007 which claims priority to U.S. Provisional Application No. 60/856,112 filed Nov. 2, 2006, the contents of which are incorporated herein by reference in their entirety.

### BACKGROUND OF THE INVENTION

1. Field of the Invention

The present invention relates to the fields of fitness, healthcare, and cosmetic surgery generally. More particularly, the invention relates to a device and method for measuring soft tissue thickness and structure with a handheld apparatus utilizing ultrasound. This device can monitor changes in adipose and muscle tissue due to changes in fitness or health. The present invention can also be used to measure total body fat by making a plurality of measurements.

2. Description of Related Art

Thickness of tissue layers and in particular adipose (fat) and muscle tissue can be important to evaluate fitness and health. There are a variety of techniques currently used to measure the thickness of the adipose layer. For example skin calipers can be used to measure the thickness of the skin fold produced when the operator pinches a subject's skin. Various equations are used to predict body density and the percent of body adipose tissue (American College of Sports Medicine (ACSM) “Guidelines For Exercise Testing And Prescription”, 53-63 (1995)). However, there are many drawbacks to this form of adipose tissue measurement. These measurements are heavily dependent on the operator, and errors and variations frequently occur. Skin fold calipers can only provide an estimate of tissue thickness and are not particularly accurate for tracking small changes.

Another means of determining body density and estimating percent body adipose tissue is a generalized measurement hydrostatic weighing. Hydrostatic weighing requires the subject to be completely immersed in water. This method of measurement could be employed before and after a liposuction procedure, which is typically impractical and costly when the goal is to monitor adipose tissue changes during the surgery. The surgeon performing liposculpture and most surgical contouring procedures requires localized measurements. Maintenance of a sterile field is problematic with such a method.

A method and apparatus is needed to efficiently and accurately measure adipose tissue. U.S. Pat. No. 5,941,825 dated Oct. 1996 by Lang et al., recognized that ultrasound could be utilized to conveniently and cost effectively measure layer thickness in an object. WO 99/65395 dated Dec. 1999 by Lang et al., builds on the previously referenced patent by using anatomical landmarks to measure changes in body adipose tissue. The aim of these two patents is to measure adipose tissue changes over time as a result of diet and exercise. However, all these patents describe single point, or what is more commonly referred to as A-mode, ultrasound transducers. There are applications where measuring the tissue thickness and structure over an extended region is valuable. For example, this can help reduce false measurements due to thin scar tissue. Medical imaging (“B-mode”) ultrasound systems can provide this data but the high cost of B-mode systems makes the B-mode systems impractical for use in fitness clubs or directly by the consumer.

There is a need for an accurate, convenient, and cost effective apparatus to measure tissue thickness and structure accurately. The present invention fulfills this need, and further provides related advantages.

### SUMMARY OF THE INVENTION

A system for accurately measuring tissue layer thickness and structure to monitor the effects of exercise or diet is disclosed.

A system to accurately measure percentage body fat and body density is also disclosed.

Additionally disclosed is a system to accurately measure adipose tissue distribution and identify superficial adipose tissue and deep adipose tissue.

Also disclosed is a control and data processing unit (e.g. portable computer), a handheld ultrasound device that contains ultrasound transducers and an integrated position sensor, and a monitor to display the information to the user.

The handheld ultrasound device can use and/or have an ultrasound transducer with a single or a plurality of ultrasound generating and detection elements to obtain an effective A-Scan (“Ultrasound in Medicine” Ed. F. A. Duck, A. C. Baker, H. C. Starritt (1997)) of the tissue structure directly below the transducer. The A-scan will show strong reflections at the interface between the various layers (e.g., skin, fat, muscle and bone). Strong ultrasound reflections occur at the interfaces due to impedance mismatch between the various materials. The A-scan signal can be analyzed by the control unit to determine the thickness of the various tissue layers (e.g., skin, fat, muscle).

An optical position sensor on the device (e.g., Agilent ADNS-2610) measures changes in the position of the ultrasound device. Alternative position sensors such as encoded rotating wheels can also be used. By monitoring the relative position of the device as the device slides on tissue, the control system can generate a two-dimensional (B-mode) image of the tissue structure. The image can be processed to accurately determine the fat-muscle interface and, if desired, calculate an average thickness over the scan area. By making multiple measurements for example, chest, waist and thigh a body fat percentage (“% body fat”) for the whole body can be calculated. In this mode the device can be used to monitor fitness programs and diet.

The device can be unconnected by a wire or cable to the control unit. The device and control unit communicate through a wireless means (e.g., RF communication, such as Bluetooth). The control unit and display can be far away from a sterile surgical field. RF communication can eliminate having to cover the control unit and cable with sterile bags. The device can be powered by a wall outlet or by batteries, which can reduce the electrical hazard.

When making total body composition measurements, the remote control unit can acquire the data from the handheld transducer and analyze the data to produce a table of tissue thickness parameters for all the anatomical points. This data can be displayed in a tabulated list or a color-coded anatomical map that can be interpreted by the user or surgeon. The display can show the change in the fat layer thickness during the course of the liposuction procedure. The user can control the display and function of the control unit through a keyboard and/or mouse interface and/or touch screen.

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 shows a sectional view taken through a variation of the handheld device which integrates an ultrasound transducer and position sensor.

FIG. 2 shows a sectional view of a variation of the handheld device with a water compartment.

FIG. 3 illustrates a variation of a method of using the handheld device.

FIG. 4 shows a single point measured signal using the present invention on a male abdomen.

FIG. 5 shows a single point measured signal using the present invention on a male bicep.

FIG. 6 shows a two-dimensional cross-section image collected by scanning the present invention along a male bicep.

### DETAILED DESCRIPTION OF THE INVENTION

A system for accurately measuring tissue layer thickness is disclosed. The system can be used to produce a map of the fat (or adipose) tissue thickness at selected or key anatomical points or locations. These measurements can be monitored and compared to track changes. The device can have a remote control and data processing unit, a handheld ultrasound transducer, and a monitor or LCD to display the information to the user.

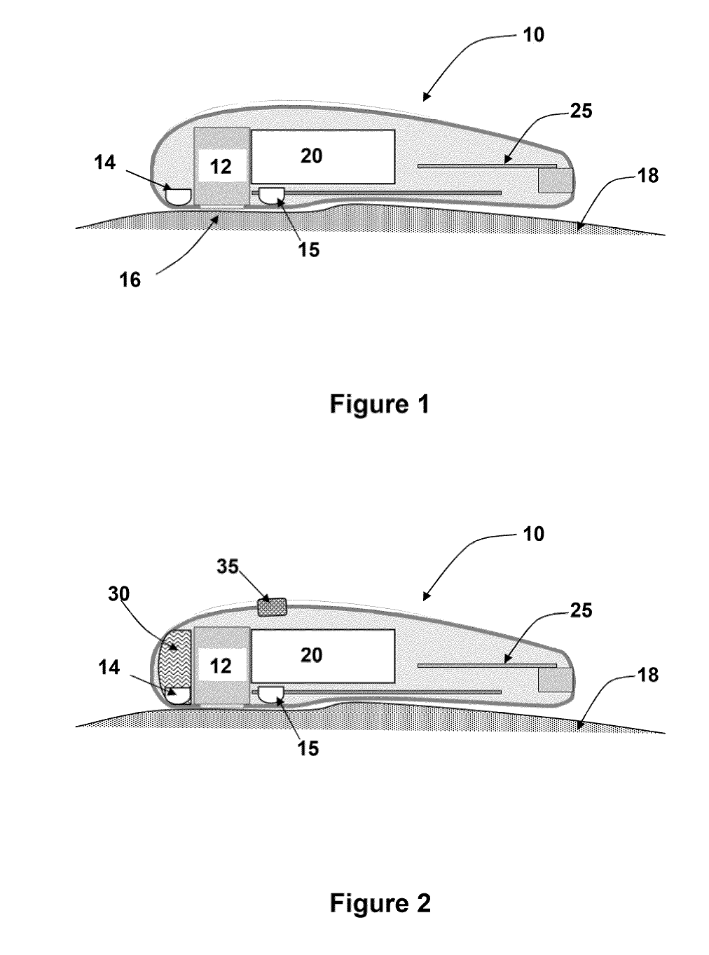

FIG. 1 shows a cross-sectional view of the handheld ultrasound measuring device 10. The device can have one or more transducers, such as an ultrasound transmitter and receiver 12. The transmitter and receiver 12 can be a single element or two separate elements. Two separate elements can reduce reflection artifacts and allow imaging closer to the transmitter element. The ultrasound transmitter and detection element can be made of any piezoelectric material. Suitable materials include ceramics (e.g., lead zirconate titanate (PZT)), or plastic (polyvinylidinedifluoride, PVDF). The operating frequency for adequate penetration and resolution in tissue can be about 500 kHz to about 10 MHz. For additional information on transducer design and operation refer to “The Physics of Medical Imaging” Ed. Steve Webb (1988), and “Ultrasound in Medicine” Ed. F. A. Duck, A. C. Baker, H. C. Starritt (1997) both of which are incorporated herein by reference. See also U.S. Pat. No. 5,699,806, titled “Ultrasound System With Nonuniform Rotation Corrector” incorporated herein by reference.

One or more optical position sensors 14 and/or 15 within or attached to the device 10 can detect changes in position as the device 10 is moved along the skin surface 18. The change in x and y position of the device is recorded and returned to the control unit. Suitable optical position sensors are produced, for example, by Agilent Technologies, Inc. of Palo Alto, Calif. (e.g. Agilent ADNS 2670) and described in numerous patent applications including U.S. Pat. No. 6,927,758, by Plot et al., which is incorporated herein by reference. The optical position sensor 14 does not have to be in direct contact with the skin. Alternative or additional position sensors can include one or more conventional encoded tracking balls or rotating wheels (e.g., such as those commonly used in a computer mouse) which require contact with the skin. A second optical position sensor 15 can be placed on the device, for example to increase the accuracy of the tracking of the position of the center of the transducer 12. From the known geometry of the device 10 the exact change in position of the transducer 12 can be calculated.

The device 10 can be powered by a battery 20 or external power cord (not shown). The measured signal can be transferred from the device 10 to a remote computer or microprocessor by wireless means 25 (e.g., Bluetooth, devices conforming to any wireless standard routinely used by computers e.g., IEEE 802.11, acoustic or optical) or cable (not shown), and/or the microprocessor can be on-board the device 10. The device 10 can also be powered and also communicate to remote computer by a USB cable (not shown).

FIG. 2 that the device 10 can have a refillable water compartment 30. When making a measurement, the user can press button 35 that can cause a small amount of water (e.g., 1-2 drops) to be released near the surface of the ultrasound transducer 12. The water can fill the gap between the transducer 12 and the skin 18 and allow efficient transmission of the acoustic energy between the tissue and the transducer 12. The surface of the ultrasound transducer 12 can be treated to be hydrophilic so that water will easily coat the surface. Instead of water a low viscosity oil or hydrogel could be used.

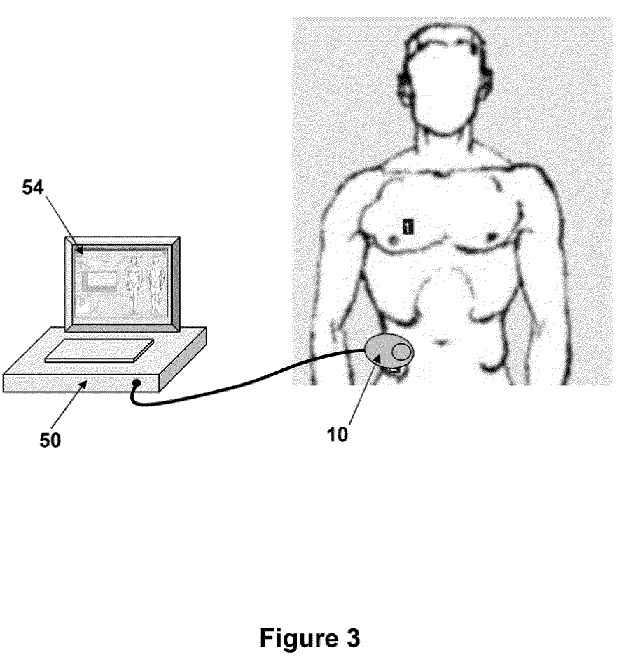

FIG. 3 illustrates how the present invention would be used to measure the local tissue structure. The measuring device 10 can be placed on the skin 18 at a point of interest. When activated, an ultrasound signal can be transmitted into the tissue and the return signal collected. The collected signal can then be communicated by cable (as shown) or by wireless means to the remote control unit 50. The control unit 50 can display the recorded waveforms and calculated thickness of relevant layers on a monitor 54. The control unit 50 can store the waveforms and information about the location of the measurement so that the user can easily monitor changes over time. The control unit 50 can be a portable computer, or PDA (e.g. HP Ipaq, Palm Pilot, etc.) The device 10 can be self-contained and a small LCD display can be on the device 10 and can display a summary of each measurement. The control unit 50 and/or the device 10 can have one or more processors and/or memory to store and/or analyze data entered by a user and/or over a network, and/or pre-programmed, and/or collected by the device 10. Wired or wireless communication can transmit measurement data between any combinations of the control unit 50, and/or device 10, and/or monitor 54.

The control unit 50 can use user-specific data such as age, height, weight, and the location of the measurement to improve the signal processing and accurately determine the tissue thickness. Interpreting standard A-scan ultrasound to identify tissue boundaries can be difficult and confusing for untrained users. By using accepted norms as a guide the control unit 50 can accurately determine the adipose tissue thickness.

The operating frequency of the transducer 12 can typically be in the range of about 500 kHz to about 10 MHz. The higher frequencies can have higher spatial resolution. The lower frequencies can have lower tissue attenuation, increasing the thickness of tissue that can be measured. The ultrasound transducer can be operated at two different frequencies. The scattered signal scales strongly with the ultrasound wavelength, the ratio of scattered signal at two frequencies can be used to determined tissue properties.

A curved transducer may be used to provide a strongly or weakly focused beam that can measure properties over a region, for example a less than 5 mm diameter region. A small diameter can reduce the blurring of layer boundaries due to non-planar layer structures. The transducer can generate the ultrasound pulse and/or measure the time history of the return acoustic signal. The collected time history signal is a measurement of the back-scattered signal as a function of depth averaged over the ultrasound beam area. The control electronics can collect and digitize the signal for further display and analysis. Additional transducers and operations can include those disclosed in “The Physics of Medical Imaging” Ed. Steve Webb (1988), “Ultrasound in Medicine” Ed. F. A. Duck, A. C. Baker, H. C. Starritt (1997), and U.S. Pat. No. 5,699,806, titled “Ultrasound System With Nonuniform Rotation Corrector”, all of which are incorporated herein by reference.

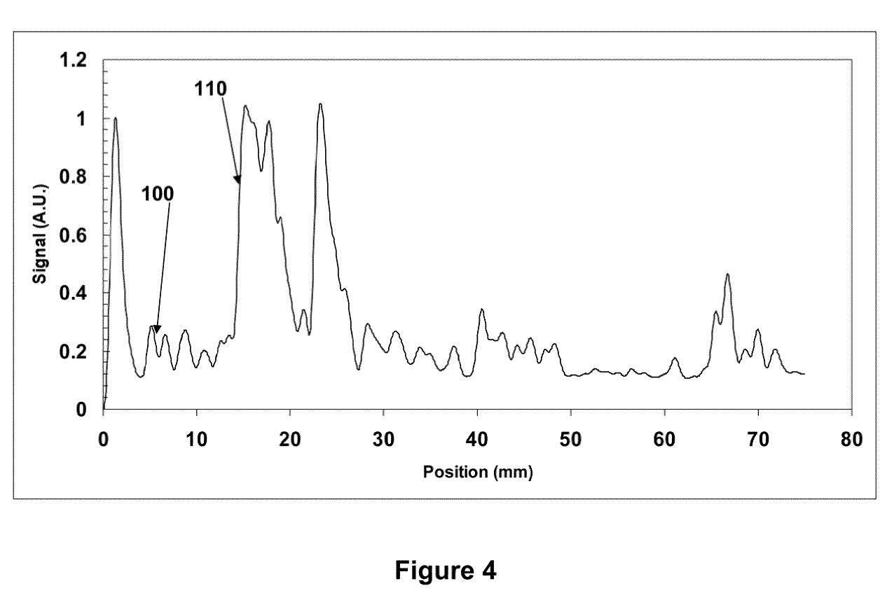

FIG. 4 shows a single A-mode measured signal using the present invention on a male abdomen. The signal peaks correspond to the interface between the device and skin 100; and fat and muscle 110. The adipose (fat) layer is located between 100 and 110 and is approximately 9.8 mm thick. Strong ultrasound reflections occur at the interfaces due to impedance mismatch between the various materials. The time history is converted to thickness by the software by using average sound speeds (c). For example, c˜1600 m/s for skin, 1400 m/s for fat, 1600 m/s for muscle, and 3500 m/s for bone (See “Ultrasound in Medicine” Ed. F. A. Duck, A. C. Baker, H. C. Starritt).

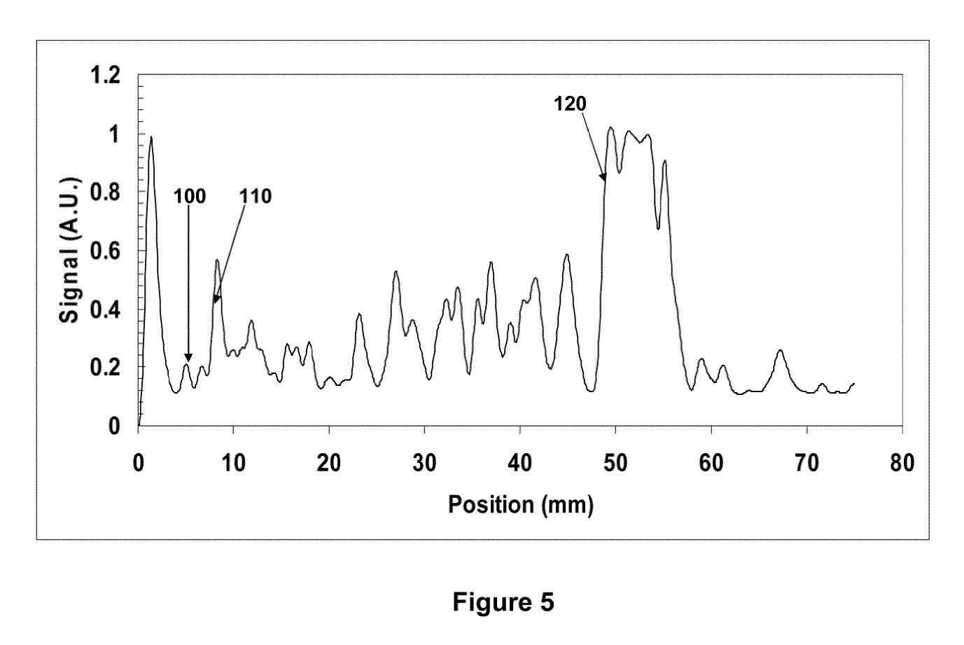

FIG. 5 shows a measured signal using the present invention on a male bicep muscle. The signal peaks correspond to the interface between the device and skin 100; fat and muscle 110 and; 120 muscle and bone. The adipose layer is located between 100 and 110 and is approximately 3.2 mm thick. The muscle layer is located between 110 and 120 and is approximately 40.8 mm thick.

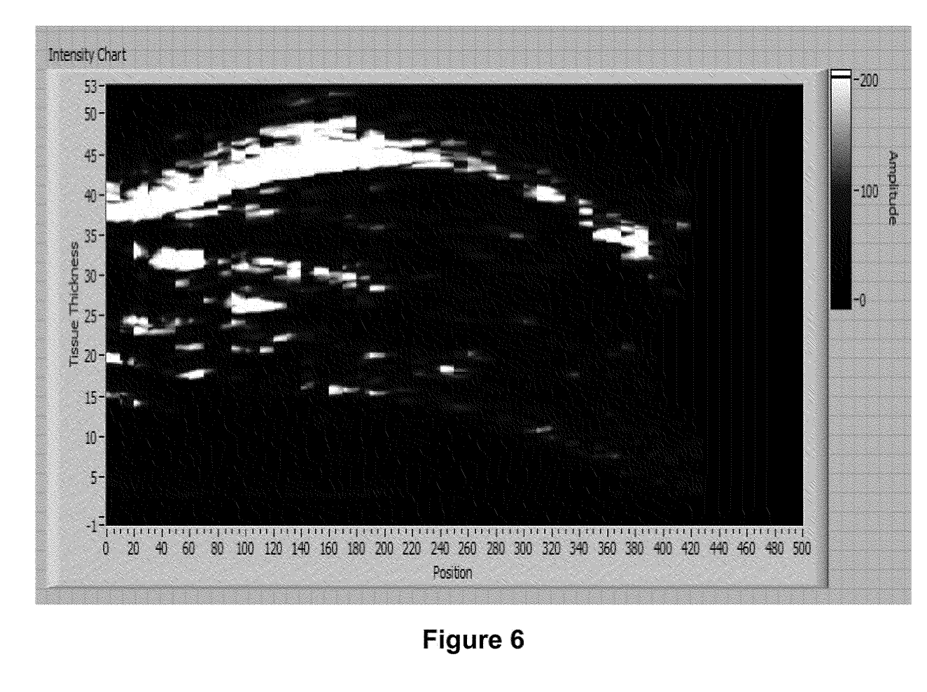

FIG. 6 shows an image of the bicep measured by scanning the device down the center of the bicep over a length of 5 inches.

The one or more processors in the control unit 50 and/or device 10 can execute control software. In order to accurately detect the interfaces, the control software can analyze the signal. The control software can determine the proper interface position, for example, based on the signal and other input information (e.g. measurement location, client weight, height, age, and gender). Strong signals can be produced at each interface due to large difference in the acoustic impedance of the different tissue types. Muscle tissue can show strong signal fluctuations. Information such as the acoustic impedance and signal fluctuations can be used to distinguish muscle from adipose tissue. The body mass index can be calculated from client weight and height and using formulas that relate percentage body fat to body mass index (e.g. Deurenberg P, Yap M, van Staveren W A. Body mass index and percent body fat. A meta analysis among different ethnic groups. Int J Obes Relat Metab Disord 1998; 22:1164-1171.). The approximate thickness of the adipose tissue can be calculated. Generally this estimated value can be 25%-50% too high for athletes. The control software can execute an algorithm into which the user can input whether the client has an athletic build or not.

The measuring device can be applied at a single point or multiple key anatomical points. By making measurements at multiple sites (e.g., at least three) the system can estimate the body density (“D”) and the percentage body fat (“% BF”). Common sites used for these estimates are as follows.

A site that can be used for taking the measurement for the triceps can be, for example, at the level of the mid-point between acromial process (boney tip of shoulder) and proximal end of the radius bone (elbow joint), on the posterior (back) surface of the arm. A site that can be used for taking the measurement for the biceps can be, for example, at the same level as for the triceps, but on the anterior (front) surface of the arm.

A site that can be used for taking the measurement for the subscapula can be, for example, about 2 cm below the lower angle of the scapula (bottom point of shoulder blade) on a line running laterally (away from the body) and downwards (at about 45 degrees).

A site that can be used for taking the measurement for the axilla can be, for example, the intersection of a horizontal line level with the bottom edge of the xiphoid process (lowest point of the breast bone), and a vertical line from the mid axilla (middle of armpit).

A site that can be used for taking the measurement for the ileac crest can be, for example, the site immediately above the iliac crest (top of hip bone), at the mid-axillary line.

A site that can be used for taking the measurement for the supraspinale can be, for example, the intersection of a line joining the spinale (front part of iliac crest) and the anterior (front) part of the axilla (armpit), and a horizontal line at the level of the iliac crest.

A site that can be used for taking the measurement for the abdominal can be, for example, about 5 cm adjacent to the umbilicus (belly-button).

A site that can be used for taking the measurement for the front thigh can be, for example, the mid-point of the anterior surface of the thigh, midway between patella (knee cap) and inguinal fold (crease at top of thigh). A site that can be used for taking the measurement for the medial calf can be, for example, the point of largest circumference on medial (inside) surface of the calf.

A site that can be used for taking the measurement for the chest can be, for example, between the axilla and nipple as high as possible on the anterior axillary fold (e.g., for males).

For example, by making a measurements at chest, abdomen, and thigh, the system can estimate the body density (D) and percentage body fat (% BF) with the following equations for males and females respectively. Anatomical locations in the formulas below refer to local skin fold thickness, which the processor in the system can calculate by multiplying the real fat tissue thickness as measured with the ultrasound transducer by a factor.

For Males:
D=1.10938−(0.0008267×sum of chest, abdominal, thigh)+(0.0000016×square of the sum of chest, abdominal, thigh)−(0.0002574×age).
Equation is based on a sample of males aged 18-61 (Jackson, A. S. & Pollock, M. L. (1978) Generalized equations for predicting body density of men. British J of Nutrition, 40: p 497-504.).

D=1.1043−(0.001327×thigh)−(0.00131×subscapular), based on a sample aged 18-26.
Sloan A W: Estimation of body fat in young men., J Appl. Physiol. (1967); 23:p 311-315.

% BF=(0.1051×sum of triceps, subscapular, supraspinale, abdominal, thigh, calf)+2.585, based on a sample of college students. Yuhasz, M. S.: Physical Fitness Manual, London Ontario, University of Western Ontario, (1974).

For Females:

D=1.0994921−(0.0009929×sum of triceps, suprailiac, thigh)+(0.0000023×square of the sum of triceps, suprailiac, thigh)−(0.0001392×age), based on a sample aged 18-55. Jackson, et al. (1980) Generalized equations for predicting body density of women. Medicine and Science in Sports and Exercise, 12:p 175-182.

D=1.0764−(0.0008×iliac crest)−(0.00088×tricep), based on a sample aged 17-25. Sloan, A. W., Burt A. J., Blyth C. S.: Estimating body fat in young women., J. Appl. Physiol. (1962); 17:p. 967-970.

% BF=(0.1548×sum of triceps, subscapular, supraspinale, abdominal, thigh, calf)+3.580, based on a sample of college students. Yuhasz, M. S.: Physical Fitness Manual, London Ontario, University of Western Ontario, (1974).

These equations can be used for thickness measurements taken with calipers and/or for fat thickness measurements made with a more accurate device (e.g., the device disclosed herein). A modified version of these equations maybe necessary when using the device disclosed herein to account for the fact that the fat layer thickness is measured and not a skin fold thickness as is common with skinfold calipers. A wide variety of other equations can be used to calculate D and % BF, for example, the other equations can offer greater accuracy but sometimes require additional information (e.g. accurate age, body type).

Software within the control unit can guide the user through the process of collecting measurements at the key anatomical sites and then display the calculated % BF and D.

This device can be used by plastic surgeons to track and monitor liposuction procedures.

The device can measure the different adipose tissue layers in the abdominal region. Superficial and deep adipose tissue form two separate regions around the abdomen. Deep adipose tissue thickness is an important indicator of heath risk.

The above descriptions and illustrations are only by way of example and are not to be taken as limiting the invention in any manner. One skilled in the art can combine disclosed for elements, structures and methods described herein for each other and substitute known equivalents.

We claim:

1. A system for accurately measuring tissue thickness and structure, comprising:
an ultrasound transducer for producing ultrasound energy and detecting reflected ultrasound;
a first position sensor for detecting changes in position as the device is moved along the skin;
a processor for analyzing data from the ultrasound transducer and the first position sensor, and for computing a displayable representation of tissue structure and measured tissue thickness; and
a display for displaying the displayable representation, wherein the display is in communication with the processor.

2. The system of claim 1, further comprising a wireless communication system configured to place the transducer and the processor in direct or indirect communication.

3. The system of claim 1, further comprising a wireless communication system configured to place the processor and the display in direct or indirect communication.

4. The system of claim 1, further comprising a fluid reservoir, wherein the system is configured to dispense a fluid from the fluid reservoir to the skin.

5. The system of claim 1, further comprising a second position sensor for detecting changes in position as the device is moved along the skin 6. The system of claim 5, wherein the first position sensor is located on a first side of the ultrasound transducer, and wherein the second position sensor is located on a second side of the ultrasound transducer.

7. The system of claim 6, wherein the first side is substantially opposite to the second side.

8. The system of claim 1, wherein the first position sensor comprises an optical sensor.

9. The system of claim 1, wherein the first position sensor comprises a rolling mechanical element.

10. The system of claim 9, wherein the first position sensor comprises a wheel.

11. The system of claim 9, wherein the first position sensor comprises a ball.

12. A method for accurately measuring tissue thickness and structure, comprising:
moving a measurement transducer along a biological surface;
tracking the location of the measurement transducer;
ultrasonically measuring the tissue thickness and/or structure with the measurement transducer;
processing with a processor the measurement data taken during the ultrasonic measurement and the tracking, and
displaying on a display processed measurement data representative of the ultrasonic measurement and the tracking.

13. The method of claim 12, further comprising communicating data wirelessly directly or indirectly from the measurement transducer to the processor.

14. The method of claim 12, further comprising communicating data wirelessly directly or indirectly from the processor transducer to the display.

15. The method of claim 12, wherein displaying the measurements comprises displaying the measurements as graphic representing an image of the tissue.

16. The method of claim 12, wherein displaying the measurements comprises displaying the measurements as substantially two-dimensional signal-to-position graph.

17. The method of claim 12, wherein moving the measurement transducer along the biological surface comprises moving the measurement transducer along a fluid between the transducer and the biological surface.

18. The method of claim 17, further comprising releasing the fluid from a reservoir contained together with the measurement transducer.

19. The method of claim 12, wherein tracking comprises tracking with an optical sensor.

20. The method of claim 12, wherein tracking comprises tracking with a rolling mechanical element.

21. The method of claim 12, wherein tracking comprises tracking with a first position sensor and a second position sensor.

22. The method of claim 12, further comprising performing liposuction, and wherein moving, tracking and measuring are concurrent with the liposuction.

23. The method of claim 12, further comprising performing liposculpture, and wherein moving, tracking and measuring are concurrent with the liposculpture.
